(19) United States
(12) Patent Application Publication
(10) Pub. No.: US 2005/0277664 A1
(43) Pub. Date: Dec. 15, 2005

(54) Monoamine neurotransmitter re-uptake inhibitor for the inhibition of beta-amyloid generation

(75) Inventors: Klaus Bornemann, Setzingen (DE); Joachim Mierau, Mainz (DE); Dagmar Kugler, Schemmerhofen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/144,481

(22) Filed: Jun. 3, 2005

(30) Foreign Application Priority Data
Jun. 4, 2004 (EP) 04013242

(51) Int. Cl. A61K 31/46

(52) U.S. Cl. 514/304

(57) ABSTRACT

The invention relates to the use of a monoamine neurotransmitter re-uptake inhibitor comprising a 2,3-disubstituted tropane moiety for the preparation of a medicament for inhibiting β-amyloid generation.

### BACKGROUND OF THE INVENTION

This application claims the benefit under 35 U.S.C. 119(a) of European Patent Application No. 04 013 242, which was filed on Jun. 4, 2004, and which, by reference, is incorporated herein in its entirety.

1. Field of the Invention

The invention relates to the use of a monoamine neurotransmitter re-uptake inhibitor comprising a 2,3-disubstituted tropane moiety optionally in the form of its physiologically acceptable acid addition salts for the preparation of a medicament for inhibiting β-amyloid generation.

2. Description of the Prior Art

Amyloid β-peptides (Aβ) are strongly aggregating peptides with approximate molecular masses of 4 kDa. The predominant forms, Aβ₄₀ and Aβ₄₂, are 40 and 42 amino acid residues in length, and are the major proteinaceous constituents of brain amyloid deposits in a variety of diseases. Aβ₄₂ is an early and central component of amyloid in diffuse and senile plaques, while Aβ₄₀ is the major peptide form in amyloid deposits in the cerebral microvasculature. Aβ₄₀ and Aβ₄₂ are derived by endoproteolysis of the larger amyloid precursor protein (APP) by the sequential activities of β-secretase at the amino-terminus, and a γ-secretase that cleaves at the C-terminus, respectively, of the Aβ domain. Alternative amino-terminal cleavage by α-secretase within the Aβ domain results in the generation of non-amyloidogenic fragments. Because Aβ peptides readily aggregate into insoluble amyloid plaques, lowering their generation is a major objective for the design of therapeutic and preventive strategies for the treatment of a variety of diseases.

### SUMMARY OF THE INVENTION

Surprisingly it has been found that a monoamine neurotransmitter re-uptake inhibitor comprising a 2,3-disubstituted tropane moiety optionally in the form of its physiologically acceptable acid addition salts dose-dependently decreases the levels of Aβ₄₂ and Aβ₄₀ that are secreted into the supernatant by an APP transfected U373 astrocytoma cell line. Furthermore, it has been found that Aβ levels are significantly decreased in APP tg mice that have been treated with a monoamine neurotransmitter re-uptake inhibitor comprising a 2,3-disubstituted tropane moiety. Accordingly, one embodiment of the current invention relates to the use of a monoamine neurotransmitter re-uptake inhibitor comprising a 2,3-disubstituted tropane moiety optionally in the form of its physiologically acceptable acid addition salts for the preparation of a medicament for the treatment or prevention of a disease or condition associated with an increased level of one or more isoforms of amyloid β peptides (Aβ), and/or with a changed ratio of levels of Aβ isoforms, and/or with the formation of plaques containing one or more amyloid β peptide isoforms in a mammal.

In a preferred embodiment the invention relates to the use of a monoamine neurotransmitter re-uptake inhibitor comprising a 2,3-disubstituted tropane moiety optionally in the form of its physiologically acceptable acid addition salts for the preparation of a medicament for the treatment or prophylaxis of diseases associated with the formation of diffuse and senile plaques. Furthermore, the invention relates to the use of a monoamine neurotransmitter re-uptake inhibitor comprising a 2,3-disubstituted tropane moiety optionally in the form of its physiologically acceptable acid addition salts for the preparation of a medicament for the treatment or prophylaxis of diseases associated with the formation of Aβ₄₀- and Aβ₄₂-containing plaques, preferably of Aβ₄₂-containing plaques.

Moreover the invention relates to the use of a monoamine neurotransmitter re-uptake inhibitor comprising a 2,3-disubstituted tropane moiety optionally in the form of its physiologically acceptable acid addition salts for the preparation of a medicament for the treatment or prophylaxis of amyloidosis associated with the formation of Aβ₄₀ and Aβ₄₂. Preferably the invention relates to the use of monoamine neurotransmitter re-uptake inhibitor comprising a 2,3-disubstituted tropane moiety optionally in the form of its physiologically acceptable acid addition salts for the preparation of a medicament for the treatment or prophylaxis of amyloidosis associated with the formation of Aβ₄₂.

In particular, the invention relates to the use of a monoamine neurotransmitter re-uptake inhibitor comprising a 2,3-disubstituted tropane moiety, optionally in the form of its physiologically acceptable acid addition salts, for the preparation of a medicament for the treatment or prophylaxis of brain amyloidosis.

Moreover, the invention relates to the use of a monoamine neurotransmitter re-uptake inhibitor comprising a 2,3-disubstituted tropane moiety, optionally in the form of its physiologically acceptable acid addition salts, for the preparation of a medicament for the non-symptomatic or disease modifying treatment of patients suffering from Alzheimer's disease (AD).

Furthermore the invention relates to the use of a monoamine neurotransmitter re-uptake inhibitor comprising a 2,3-disubstituted tropane moiety, optionally in the form of its physiologically acceptable acid addition salts, for the preparation of a medicament for helping to prevent or delay the onset of AD, for treating patients with mild cognitive impairment (MCI), and preventing or delaying the onset of AD in those patients who would otherwise be expected to progress from MCI to AD, for treating Down's syndrome, for treating Hereditary Cerebral Hemorrhage with Amyloidosis of the Dutch Type, for treating cerebral beta-amyloid angiopathy and preventing its potential consequences such as single and recurrent lobar hemorrhages, for treating other degenerative dementias, including dementias of mixed vascular and degenerative origin, for treating dementia associated with Parkinson's disease, dementia associated with progressive supranuclear palsy, dementia associated with cortical basal degeneration, and diffuse Lewy body type AD.

### DESCRIPTION OF THE DRAWINGS

FIG. 1 is a graph of a dose response curve showing an increasing inhibition of Aβ₄₀ with an increasing concentration of the compound of Formula IA of the present invention.

FIG. 2 is a graph of a dose response curve showing an increasing inhibition of Aβ₄₂ with an increasing concentration of the compound of Formula IA of the present invention.

FIG. 3 is a graph of the results of an in vivo test of the compound of formula IA that revealed a statistically significant reduction of the Aβ₄₀ levels.

### DETAILED DESCRIPTION OF THE INVENTION

As a rule, the monoamine neurotransmitter re-uptake inhibitor comprising a 2,3-disubstituted tropane moiety are those that are disclosed by International Patent Applications WO 93/09814 and WO 97/30997, which are, by reference, incorporated herein in their entireties.

Preferably the monoamine neurotransmitter re-uptake inhibitor comprising a 2,3-disubstituted tropane moiety are compounds of the general formula (I) 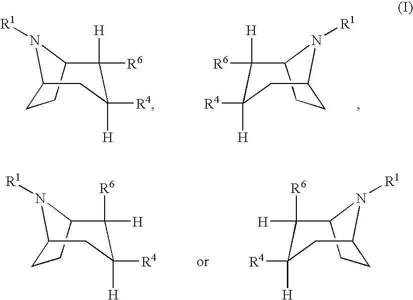
or a pharmaceutical acceptable addition salt thereof, or the N-oxide thereof, wherein
R¹ is hydrogen, alkyl, alkenyl, alkynyl, cycloalkyl, cycloalkylalkyl, or 2-hydroxyethyl;
R⁶ is
   CH₂—X—R³, wherein
      X is O, S, or NR′; wherein
         R′ is hydrogen or alkyl; and
         R³ is alkyl, alkenyl, alkynyl, cycloalkyl, cycloalkylalkyl, or —CO-alkyl;
   heteroaryl, which may be substituted one or more times with
      alkyl, cycloalkyl, or cycloalkylalkyl;
      phenyl, which may be substituted one or more times with substituents selected from the group consisting of: halogen, CF₃, CN, alkoxy, alkyl, alkenyl, alkynyl, amino, nitro, and heteroaryl;
      phenylphenyl;
      pyridyl, which may be substituted one or more times with substituents selected from the group consisting of halogen, CF₃, CN, alkoxy, alkyl, alkenyl, alkynyl, amino, nitro, and heteroaryl;
      thienyl, which may be substituted one or more times with substituents selected from the group consisting of halogen, CF₃, CN, alkoxy, alkyl, alkenyl, alkynyl, amino, nitro, and heteroaryl; or
      benzyl, which may be substituted one or more times with substituents selected from the group consisting of halogen, CF₃, CN, alkoxy, alkyl, alkenyl, alkynyl, amino, nitro, and heteroaryl; or
   (CH₂)ₙCO₂R⁷, COR⁷, or CH₂R⁸, wherein
      R⁷ is
         alkyl, cycloalkyl, or cycloalkylalkyl;
         Phenyl, which may be substituted one or more times with substituents selected from the group consisting of: halogen, CF₃, CN, alkoxy, alkyl, alkenyl, alkynyl, amino, nitro, and heteroaryl;
         phenylphenyl;
         pyridyl, which may be substituted one or more times with substituents selected from the group consisting of: halogen, CF₃, CN, alkoxy, alkyl, alkenyl, alkynyl, amino, nitro, and heteroaryl;
         o-thienyl, which may be substituted one or more times with substituents selected from the group consisting of: halogen, CF₃, CN, alkoxy, alkyl, alkenyl, alkynyl, amino, nitro, and heteroaryl; or
         benzyl;
      n is 0 or 1; and
      R⁸ is
         O-phenyl, which may be substituted one or more times with substituents selected from the group consisting of: halogen, CF₃, CN, alkoxy, alkyl, alkenyl, alkynyl, amino, nitro, and heteroaryl;
         O—CO-phenyl that may be substituted one or more times with substituents selected from the group consisting of: halogen, CF₃, CN, alkoxy, alkyl, alkenyl, alkynyl, amino, nitro, and heteroaryl;
         CH═NOR³, wherein R³ is o-hydrogen; o-alkyl, cycloalkyl, cycloalkylalkyl, alkenyl, alkynyl, or aryl, all of which may be substituted with —COOH;
         —COO-alkyl;
         —COO-cycloalkyl; or
         phenyl that may be substituted one or more times with substituents selected from the group consisting of: halogen, CF₃, CN, alkyl, cycloalkyl, alkoxy, cycloalkoxy, alkenyl, alkynyl, amino, and nitro; and
R⁴ is phenyl, 3,4-methylenedioxyphenyl, benzyl, naphthyl, or heteroaryl, all of which may be substituted one or more times with substituents selected from the group consisting of: halogen, CF₃, CN, alkoxy, cycloalkoxy, alkyl, cycloalkyl, alkenyl, alkynyl, amino, nitro, and heteroaryl.

In a special embodiment of the compound of general formula I, R⁶ is:
1,2,4-oxadiazol-3-yl, which may be substituted in the 5 position with:
   alkyl, cycloalkyl, or cycloalkylalkyl;
   phenyl, which may be substituted one or more times with substituents selected from the group consisting of: halogen, CF₃, CN, alkoxy, alkyl, alkenyl, alkynyl, amino, nitro, and heteroaryl;
   phenylphenyl; or
   benzyl, which may be substituted one or more times with substituents selected from the group consisting of: halogen, CF₃, CN, alkoxy, alkyl, alkenyl, alkynyl, amino, nitro, and heteroaryl; or
1,2,4-oxadiazol-5-yl, which may by substituted in the 3 position with
   alkyl, cycloalkyl, or cycloalkylalkyl;
   phenyl, which may be substituted one or more times with substituents selected from the group consisting of: halogen, CF₃, CN, alkoxy, alkyl, alkenyl, alkynyl, amino, nitro, and heteroaryl; phenylphenyl;
   benzyl, which may be substituted one or more times with substituents selected from the group consisting of: halogen, CF₃, CN, alkoxy, alkyl, alkenyl, alkynyl, amino, nitro, and heteroaryl;
   pyridyl, which may be substituted one or more times with substituents selected from the group consisting of: halogen, CF₃, CN, alkoxy, alkyl, alkenyl, alkynyl, amino, nitro, and heteroaryl; or
   thienyl, which may be substituted one or more times with substituents selected from the group consisting of: halogen, CF₃, CN, alkoxy, alkyl, alkenyl, alkynyl, amino, nitro, and heteroaryl.

In a further special embodiment of the compound of general formula (I), R⁶ is CH₂—X—R³, wherein X is O, S, or NR′; wherein R′ is hydrogen or alkyl, and R³ is alkyl, alkenyl, alkynyl, cycloalkyl, cycloalkylalkyl, or —CO-alkyl.

In a still further embodiment of the compound of general formula (I), R⁶ is CH═NOR³; wherein R³ is hydrogen; alkyl, cycloalkyl, cycloalkylalkyl, alkenyl, alkynyl, or aryl, all of which may be substituted with —COOH; —COO-alkyl; —COO-cycloalkyl; or phenyl, which may be substituted one or more times with substituents selected from the group consisting of: halogen, CF₃, CN, alkyl, cycloalkyl, alkoxy, cycloalkoxy, alkenyl, alkynyl, amino, and nitro.

In a further special embodiment of the compound of general formula (I), R⁴ is phenyl, which is substituted once or twice with substituents selected from the group consisting of: halogen, CF₃, CN, alkoxy, cycloalkoxy, alkyl, cycloalkyl, alkenyl, alkynyl, amino, nitro, and heteroaryl.

In a more special embodiment, R⁴ is phenyl substituted once or twice with chlorine.

In a further special embodiment, the tropane derivative having dopamine reuptake inhibitor activity is a (1 R, 2R, 3S)-2,3-disubstituted tropane derivative of formula I.

In a still further embodiment, the tropane derivative having dopamine reuptake inhibitory activity is a compound of general formula I, wherein R⁶ is —CH₂—X—R³, wherein X is O or S, and R³ is methyl, ethyl, propyl, or cyclopropylmethyl; —CH═NOR³; wherein R³ is hydrogen or alkyl; or 1,2,4-oxadiazol-5-yl which may by substituted in the 3 position with alkyl.

In a still further embodiment, the tropane derivative having dopamine reuptake inhibitory activity is a compound of general formula I wherein R¹ is hydrogen, methyl, ethyl, or propyl.

In a still further embodiment, the tropane derivative having dopamine reuptake inhibitory activity is a compound of general formula I, wherein R⁴ is 3,4-dichlorophenyl.

More preferably those monoamine neurotransmitter re-uptake inhibitor comprising a 2,3-disubstituted tropane moiety are compounds of formula (I1) 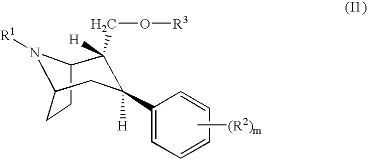
wherein
R¹ represents a hydrogen atom or a C₁₋₆ alkyl group, preferably a hydrogen atom, a methyl or an ethyl group;
R² each independently represents a halogen atom, or a CF₃, or cyano group, preferably a fluorine, chlorine, or bromine atom;
R³ represents a hydrogen atom, or a C₁₋₆ alkyl, or C₃₋₆-cycloalkyl-C₁₋₃-alkyl group, preferably a methyl, ethyl or n-propyl group; and
m is 0 or an integer from 1 to 3, preferably 1 or 2;
   or a tautomer, a pharmaceutically acceptable salt, solvate, or physiologically functional derivative thereof.

As used herein, the expression “Cₗ₋₆ alkyl” includes methyl and ethyl groups, and straight-chained and branched propyl, butyl, pentyl and hexyl groups. Particular alkyl groups are methyl, ethyl, n-propyl, isopropyl, and t-butyl.

The expression “C₃-₆ cycloalkyl” as used herein includes cyclic propyl, butyl, pentyl and hexyl groups, such as cyclopropyl and cyclohexyl.

The term “halogen” as used herein includes fluorine, chlorine, bromine, and iodine, of which fluorine and chlorine are preferred.

The term “physiologically functional derivative” as used herein includes derivatives obtained from the compound of formula (I) under physiological conditions, these are, for example, N-oxides, which are formed under oxidative conditions.

The term “pharmaceutically acceptable acid addition salt” as used herein includes those salts that are selected from among the acid addition salts formed with hydrochloric acid, hydrobromic acid, sulphuric acid, phosphoric acid, methanesulphonic acid, acetic acid, fumaric acid, succinic acid, lactic acid, citric acid, tartaric acid, and maleic acid, the salts obtained from hydrochloric acid, hydrobromic acid, sulphuric acid, phosphoric acid, and acetic acid being particularly preferred. The salts of citric acid are of particular significance.

In a special embodiment, the tropane derivative having dopamine reuptake inhibitor activity is a compound of the general formula (I) selected from: (
1R,2R,3S)-2-(3-Cyclopropyl-1 2,4-oxadiazol-5-yl)-3-(4-fluorophenyl) tropane; (
1R,2R,3S)-2-(3-Phenyl-1,2,4-oxadiazol-5-yl)-3-(4-fluorophenyi) tropane; (
1R,2R,3S)-2-(3-Phenyl-1,2,4-oxadiazol-5-yl)-3-(4-methylphenyl)-tropane; (
1R,2R,3S)-2-(3-Benyl-1,2,4-oxadiazol-5-yl)-3-(4-fluorophenyl) tropane; (
1R,2R,3S)-2-(3-(4-Phenyl-phenyl)-1,2,4-oxadiazol-5-yl)-3-(4-fluorophenyl) tropane; (
1R,2R,3S)-2-(3-Phenyl-1,2,4-oxadiazol-5-yl)-3-(2-naphthyl) tropane; (
1R,2R,3S)-3-(3,4-Dichlorophenyl) tropane-2-aldoxime; (
1R,2R,3S)-3-(3,4-Dichlorophenyl)-tropane-2-O-methyl-aldoxime; (
1R,2R,3S)-3-(3,4-Dichlorophenyl)tropane-2-O-benzyl-aldoxime; (
1R,2R,3S)-3-(3,4-Dichlorophenyl) tropane-2-O-ethoxycarbonylmethyl-aldoxime; (
1R,2R,3S)-3-(3,4-Dichlorophenyl) tropane-2-O-methoxycarbonylmethyl-aldoxime; (
1R,2R,3S)-3-(3,4-Dichlorophenyl)tropane-2-O-(1-ethoxycarbonyl-1,1-dimethyl-methyl)-aldoxime; (
1R,2R,3S)-3-(3,4-Dichlorophenyl) tropane-2-O-carboxymethyl-2-aldoxime; (
1R,2R,3S)-N-Normethyl-3-(3,4-dichlorophenyl) tropane-2-O-methyl-aldoxime; (
1R,2R,3S)-N-Normethyl-3-(3,4-dichlorophenyl) tropane-2-O-benzyl-aldoxime; (
1R,2R,3S)-3-(4-Methylphenyl) tropane-2-O-methyl-aldoxime; (
1R,2R,3S)-3-(3,4-Dichlorophenyl)tropane-2-O-(1,1-dimethylethyl)-aldoxime; (
1R,2R,3S)-3-(4-Chlorophenyl) tropane-2-O-aldoxime; (
1R,2R,3S)-3-(4-Chlorophenyl) tropane-2-O-methylaldoxime hydrochloride; (
1R,2R,3S)-3-(4-Chlorophenyl)tropane-2-O-methoxycarbonylmethyl-aldoxime; (
1R,2R,3S)-3-(3,4-Dichlorophenyl) tropane-2-O-(2-propynyl)-aldoxime; (
1R,2R,3S)-3-(3,4-Dichlorophenyl)tropane-2-O-(2-methylpropyl)-aldoxime; (
1R,2R,3S)-3-(3,4-Dichlorophenyl)tropane-2-O-cyclopropylmethyl-aldoxime; (
1R,2R,3S)-3-(3,4-Dichlorophenyl) tropane-2-O-ethyl-aldoxime; (
1R,2R,3S)-2-Methoxymethyl-3-(3,4-dichlorophenyl)-tropane; (
1R,2R,3S)-2-Isopropoxymethyl-3-(3,4-dichlorophenyl)-tropane; (
1R,2R,3S)-2-Ethoxymethyl-3-(3,4-dichlorophenyl)-tropane; (
1R,2R,3S)-2-Ethoxymethyl-3-(3,4-dichlorophenyl)-nortropane; (
1R,2R, S)-2-Cyclopropylmethyloxymethyl-3-(3,4-dichlorophenyl)-tropane; (
1R,2R,3S)-2-Methoxymethyl-3-(4-chlorophenyl)-tropane; (
1R,2R,3S)-N-Normethyl-2-methoxymethyl-3-(4-chlorophenyl)-tropane; (
1R,2R,3S)-2-Ethoxymethyl-3-(4-chlorophenyl)-tropane; (
1R,2R,3S)-N-Normethyl-2-methoxymethyl-3-(3,4-dichlorophenyl)-tropane; (
1R,2R,3S)-N-Normethyl-2-ethoxymethyl-3-(3,4-dichlorophenyl)-tropane; (
1R,2R,3S)-N-Normethyl-2-ethoxymethyl-3-(4-chlorophenyl)-tropane; (
1R,2R,3S)-N-Normethyl-2-cyclopropylmethyloxymethyl-3-(4-chlorophenyl)-tropane; (
1R,2R,3S)-2-Cyclopropylmethyloxymethyl-3-(4-chlorophenyl)-tropane; (
1R,2R,3S)-2-Ethylthiomethyl-3-(3,4-dichlorophenyl)-tropane; (
1R,2R,3S)-2-Hydroxymethyl-3-(4-fluorophenyl) tropane; (
1R,2R,3S)-2-Hydroxymethyl-3-(3,4-dichlorophenyl) tropane; (
1R,2R,3S)-N-Normethyl-N-(tert-butoxycarbonyl)-2-hydroxymethyl-3-(3,4-dichlorophenyl) tropane; (
1R,2R,3S)-2-Hydroxymethyl-3-(4-chlorophenyl) tropane; (
1R,2R,3S)-2-(3-(2-Furanyl)-1,2,4-oxadiazol-5-yl)-3-(3,4-dichlorophenyl)-tropane; (
1R,2R,3S)-2-(3-(3-Pyridyl)-1,2,4-oxadiazol-5-yl)-3-(3,4-dichlorophenyl)-tropane; (
1R,2R,3S)-N-Normethyl-N-allyl-2-(3-(4-pyridyl)-1,2,4-oxadiazol-5-yl)-3-(3,4-dichlorophenyl)-tropane; (
1R,2R,3S)-N-Normethyl-N-ethyl-2-(3-(4-pyridyl)-1,2,4-oxadiazol-5-yl)-3-(3,4-dichlorophenyl)-tropane; (
1R,2R,3S)-N-Normethyl-N-(2-hydroxyethyl)-2-(3-(4-pyridyl)-1,2,4-oxadiazol-5-yl)-3-(3,4-dichlorophenyl)-tropane; (
1R,2R,3S)-N-Normethyl-2-(3-(4-pyridyl)-1,2,4-oxadiazol-5-yl)-3-(3,4-dichlorophenyl)-tropane; (
1R,2R,3S)-N-Normethyl-N-allyl-2-(3-(3-pyridyl)-1,2,4-oxadiazol-5-yl)-3-(3,4-dichlorophenyl)-tropane; (
1R,2R,3S)-N-Normethyl-N-allyl-2-(3-(2-pyridyl)-1,2,4-oxadiazol-5-yl)-3-(3,4-dichlorophenyl)-tropane; (
1R,2R,3S)-2-(3-(2-Thienyl)-1,2,4-oxadiazol-5-yl)-3-(4-chlorophenyl)-tropane; (
1R,2R,3S)-2-(3-(2-Thienyl)-1,2,4-oxadiazol-5-yl)-3-(3,4-dichlorophenyl)-tropane; (
1R,2R,3S)-2-(3-(4-Pyridyl)-1,2,4-oxadiazol-5-yl)-3-(3,4-dichlorophenyl)-tropane; (
1R,2R,3S)-2-(3-(2-Pyridyl)-1,2,4-oxadiazol-5-yl)-3-(3,4-dichlorophenyl)-tropane; (
1R,2R,3S)-2-(3-(4-Pyridyl)-1,2,4-oxadiazol-5-yl)-3-(4-chlorophenyl)-tropane; (
1R,2R,3S)-2-(3-(3-Pyridyl)-1,2,4-oxadiazol-5-yl)-3-(4-chlorophenyl)-tropane; (
1R,2R,3S)-2-(3-2-Pyridyl)-1,2,4-oxadiazol-5-yl)-3-(4-chlorophenyl)-tropane; (
1R,2R,3S)-2-(3-Phenyl-1,2,4-oxadiazol-5-yl)-3-(4-fluorophenyl)-tropane; (
1R,2R,3S)-2-(3-Phenyl-1,2,4-oxadiazol-5-yl)-3-(4-methylphenyl)-tropane; (
1R,2R,3S)-2-(3-Benzyl-1,2,4-oxadiazol-5-yl)-3-(4-fluorophenyl)-tropane; (
1R,2R,3S)-2-(3-(4-Phenylphenyl)-1,2,4-oxadiazol-5-yl)-3-(4-fluorophenyl)-tropane; (
1R,2R,3S)-2-(3-Phenyl-1,2,4-oxadiazol-5-yl)-3-(2-naphthyl)-tropane; (
1R,2R,3S)-2-(4-Chlorophenoxy-methyl)-3-(4-fluorophenyl)-tropane; (
1R,2R,3S)-2-(4-Chlorophenoxy-methyl)-3-(4-fluorophenyl)-tropane; (
1R,2R,3S)-2-(4-Chlorophenoxy-methyl)-3-(3,4-dichlorophenyl)-tropane; (
1R,2R,3S)-2-(4-Chlorophenoxy-methyl)-3-(4-methylphenyl)-tropane; (
1R,2R,3S)-2-(4-Benzoyloxy-methyl)-3-(4-fluorophenyl)-tropane; (
1R,2R,3S)-2-Carbomethoxy-3-(2-naphthyl)-tropane; (
1R,2R,3S)-2-Carbomethoxy-3-(3,4-dichlorophenyl)-tropane; (
1R,2R,3S)-2-Carbomethoxy-3-benzyl-tropane; (
1R,2R,3S)-2-Carbomethoxy-3-(4-chlorophenyl)-tropane; (
1R,2R,3S)-2-Carbomethoxy-3-(4-methylphenyl)-tropane; (
1R,2R,3S)-2-Carbomethoxy-3-(1-naphthyl)-tropane; (
1R,2R,3S)-2-Carbomethoxy-3-(4-phenylphenyl)-tropane; (
1R,2R,3S)-2-Carbomethoxy-3-(4-t-butyl-phenyl)-tropane; (
1R,2R,3S)-2-(4-Fluoro-benzoyl)-3-(4-fluorophenyl)-tropane; or a pharmaceutically acceptable addition salt thereof.

Most preferred are the compounds of formulae (IA) and (IB) 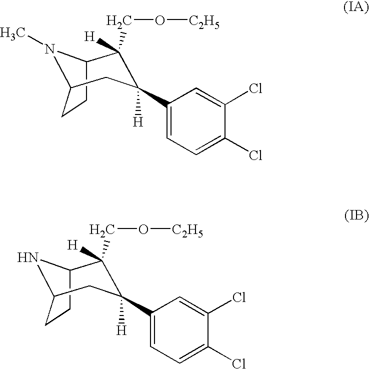
or pharmaceutically acceptable salts thereof, in particular the citrates thereof.

Accordingly, one embodiment of the current invention relates to the use of a monoamine neurotransmitter re-uptake inhibitor comprising a 2,3-disubstituted tropane moiety, optionally in the form of its physiologically acceptable acid addition salts, for the preparation of a medicament for the treatment or prevention of a disease or condition associated with an increased level of one or more isoforms of amyloid β peptides (Aβ) and/or with a changed ratio of levels of Aβ isoforms and/or with the formation of plaques containing one or more amyloid β, peptide isoforms in a mammal. Preferably the invention relates to the use of compound of formula IA for the preparation of a medicament for lowering the level of Aβ₄₂.

In a preferred embodiment of the invention relates to the use of formula IA for the preparation of a medicament for the treatment or prophylaxis of diseases associated with the formation of diffuse and senile plaques.

Furthermore, the invention relates to the use of the compound of formula IA for the preparation of a medicament for the treatment or prophylaxis of diseases associated with the formation of Aβ₄₀- and Aβ₄₂-containing plaques. Preferably, the invention relates to the use of the compound of formula IA for the preparation of a medicament for the treatment or prophylaxis of diseases associated with the formation of Aβ₄₂-containing plaques.

Moreover the invention relates to the use of the compound of formula IA for the preparation of a medicament for the treatment or prophylaxis of amyloidosis associated with the formation of Aβ₄₀, and Aβ₄₂. Preferably the invention relates to the use of the compound of formula IA for the preparation of a medicament for the treatment or prophylaxis of amyloidosis associated with the formation of Aβ₄₂.

In particular the invention relates to the use of the compound of formula IA for the preparation of a medicament for the treatment or prophylaxis of brain amyloidosis.

Furthermore the invention relates to the use of the compound of formula IA for the preparation of a medicament for the treatment or prophylaxis of vascular amyloidosis and age related amyloidosis.

Moreover, the invention relates to the use of the compound of formula IA for the preparation of a medicament for the treatment of patients suffering from mild to moderate dementia of the Alzheimer type (DAT). Furthermore the invention relates to the use of the compound of formula IA for the preparation of a medicament for the prophylactic treatment of patients identified to have a high risk for developing dementia of the Alzheimer type.

Moreover, the invention relates to the use of the compound of formula IA for the preparation of a medicament for the treatment of patients suffering from mild cognitive impairment (MCI) or age associated memory impairment (AAMI).

Furthermore the invention relates to the use of the compound of formula IA for the preparation of a medicament for the prophylactic treatment of mild cognitive impairment (MCI) or age associated memory impairment (AAMI).

Methods:

Preferably the assay is carried out as follows.

Cell culture and drug treatment: U373 astrocytoma cells expressing human wtAPP695 were used to test the compound of formula IA for Aβ lowering potential. Cells were cultured in 96 well plates in DMEM medium, additionally supplemented with 10% FCS and 1% glutamine, until they have grown to a confluent cell layer. The cells were then incubated for 17 hours in the presence of the compound of formula IA in DMEM medium. Afterwards, 100 μl of the supernatant had been removed and measured with the ELISA as described below to determine the Aβ₄₂ peptide concentrations. The cells were washed, incubated again for 4 hours with the compound, before measuring the Aβ₄₀ levels. AlamarBlue assays (Serotec, Oxford, UK) were conducted to determine cytotoxicity.

Sandwich ELISA for Aβ:

Monoclonal 6E10 against Aβ1-17 (Signet Laboratories, Inc., Dedham, Mass., USA) was used to capture Aβ₄₀; SGY 3160 against Aβ1-16 (Mayo Medical Ventures, Rochester, Minn., USA) to capture Aβ₄₂. Both antibodies were diluted in PBS at a concentration of 8 μg/ml to coat a 96 well plate. Blocking was completed with 1% Block ACE (blocking reagent) (Dainippon Seiyaku, Asaka, Japan) in PBS for 2 hrs. The plates were then washed with PBST and the cell supernatants, diluted 1:1.5 in EC buffer (0.1 M NaH2PO4, 0.1 M Na2HPO4, 2 mM EDTA, 0.4 M NaCl, 0.2% BSA, 0.05% CHAPS, 0.4% Block ACE, 0.05% NaN3 pH 7.0) have been added into the wells, before the plates were stored at 4° C. over night. Detector antibodies (alkaline phosphatase-coupled ROβ₄₀ and ROβ₄₂ against Aβ₄₀ and Aβ₄₂, respectively), were loaded onto the wells at 0.1 μg/ml in ACE Block for 2 hrs. The reporter system used was the Tropix ELISA-Light chemiluminescent detection system (Applied Biosystems (Tropix), Bedford, Mass., USA).

Animal Studies:

APPtg mice at 3 to 4 months of age were used. A compound of formula (IA) was prepared and administered in a suspension of 0.5% Tylose solution. The acetylcholinesterase inhibitor, Donepezil, had been ordered from APIN chemicals (Code 32039d).

The compound of formula (IA) and Donepezil were administered per os, using an Acrofirm needle (model 1464LL). Controls were treated with Tylose only. Each group consisted of 12 or 13 mice with equal numbers of each sex. In the short term study, the animals were treated for the time period of 2.5 days. Twice a day, a dose of 3 mg/kg was applied with interruption times of 11.5-12.5 hours. On the last day (day of sacrifice), one dose of 3 mg/kg was administered and the mice were sacrificed 5.5 hours later. In a two weeks study, 3 mg/kg of compound of formula IA and 3.3 mg/kg of Donepezil were administered once a day. In a second long term four weeks study, 3 mg/kg/day of compound of formula IA were administered, subdivided into two subdoses, with an interruption time of 10-12 hours during the day. Each version of the in vivo experiments has been performed once.

The murine brains were rapidly removed from the skull and divided along the medial fissure. The cerebellum was removed before each half was quickly frozen down on a metal plate that had been cooled down on dry ice. Brains were placed in Eppendorf tubes, frozen in liquid nitrogen, and stored at −80° C. until needed for Aβ extractions or compound measurements.

Aβ Extraction:

Brains were thawed on ice. Mouse hemibrains were extracted in a homogenisation buffer consisting of 20 mM Tris (pH 8.5), 0.2% Triton X-100, and complete proteinase inhibitor with EDTA (Roche Diagnostics GmbH, Mannheim, Germany). The brains were homogenized in a volume (ml) 5 times the weight of the brain (mg) using a 2 ml Douncer Homogenator (B. Braun, Melsungen, Germany). This was carried out 12 times with a Stempel L, followed by a Stempel S. The homogenates were then ultracentrifuged in Ultracentrifuge tubes (Beckman, CA, USA) at 200.000 g (UZ Sorvall RC 120 GX, KENDRO Laboratories Products GmbH, Hanau, Germany) at 4° C. for 1 hour. The post nuclear supernatants containing the soluble Aβ were collected and measured in a Sandwich ELISA (s.a.).

Statistical Analyses:

Statistical analyses of data was done by one-sided t tests for differences between treatment and control group to determine the p values.

Results:

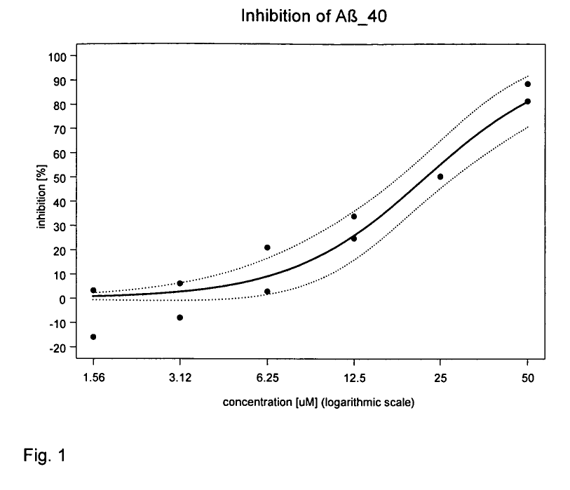

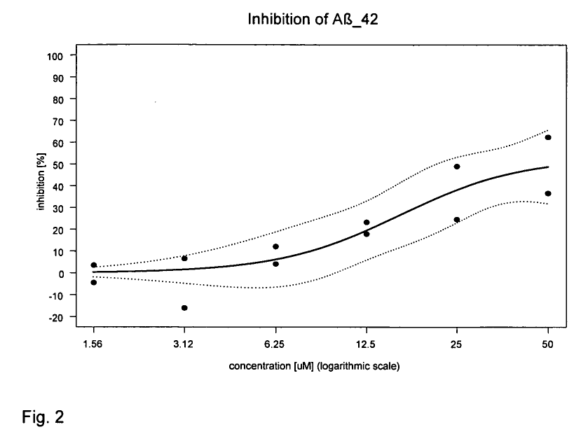

The compound of formula IA has been tested in an Aβ secretion assay. In this particular in vitro assay, the astrocytoma cell line U373 that stably over-expresses wild-type human amyloid precursor protein (APP) has been exposed to this compound. APP is proteolytically cleaved by 2 enzymes, BACE and γ secretase, to generate the Aβ peptides. Because of a flexible APP cleavage site of γ secretase, several Aβ isoforms are generated, majorly Aβ₄₀ and Aβ₄₂. The rate of Aβ generation/secretion into the medium in the presence or absence of the compound of formula IA in different concentrations has been measured by ELISA (Table 1). The % inhibition (−) or stimulation (+) has been determined in 2 independent experiments. The dose-response curves are shown in FIGS. 1 and 2.

TABLE 1
Inhibition (−) or Stimulation (+) of Aβ Secretion
in the Presence of Formula IA at Different Concentrations
in 2 Independent Experiments

| Concentration of | Aβ₄₂ Secretion | Aβ₄₀ Secretion |
|---|---|---|
| Formula IA | [%] | [%] |
| [μM] | (Exp. 1; Exp. 2) | (Exp. 1; Exp. 2) |
| 1.56 | −3.5; +4.5 | −3.2; +16 |
| 3.12 | −6.5; +16.1 | −6.1; +8.0 |
| 6.25 | −4.1; −12.1 | −20.9; −2.8 |
| 12.5 | −17.8; −23.2 | −33.9; −24.8 |
| 25 | −24.5; −48.9 | −50.1; −50.3 |
| 50 | −26.6; −62.3 | −81.3; −88.5 |

The generation/secretion of both Aβ-isoforms are inhibited by the compound of formula 1A. Inhibition of Aβ₄₀ by this compound is more pronounced, compared to the Aβ₄₂ isoform (see FIGS. 1, 2). In both cases, Aβ inhibition has been found to be dose-dependent.

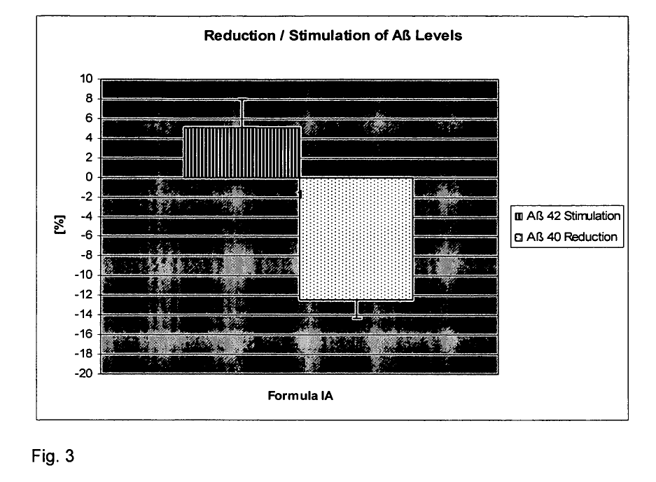

In the short term in vivo experiment, the compound of formula IA revealed a statistically significant reduction of the Aβ₄₀ levels by 12.4% (p=0.0003) (see FIG. 3 and Table 2). Aβ₄₂ levels were slightly increased by 5.1% (p=0.9136). After a two weeks treatment, compound of formula IA revealed a significant reduction of the cerebral Aβ₄₀ levels by 18.6% (p=0.024) and Aβ₄₂ levels by 16.3% (p=0.0096) (see Table 2). Donepezil slightly increased Aβ₄₀ levels by 9.6% (p=0.022) and Aβ₄₂ levels were decreases by 7.6% (p=0.402). The administration of compound of formula IA in the four weeks study revealed a significant reduction of Aβ₄₀ by 17.2% and 27.4% reduction for Aβ42. Due to the testing strategy (dose response), the reduction of Aβ40 in this experiment could not be shown to be significant.

TABLE 2
Reduction (−) and/or Stimulation (+) of cerebral Aβ levels

| | Experimental | Level of Aβ40 | Level of Aβ42 |
|---|---|---|---|
| Compound | Design | [%] | [%] |
| Formula IA | 2.5 days | −12.4 ± 1.9** | +5.1 ± 3.0 |
| | 2 × 3.0 mg/kg day 1, 2 | | |
| | 1 × 3.0 mg/kg day 3 | | |
| Formula IA | 2 weeks | −18.6 ± 7.1* | −16.3 ± 3.7** |
| | 1 × 3.0 mg/kg/day | | |
| Donepezil | 2 weeks | +9.6 ± 3.1* | −7.6 ± 7.1 |
| | 3.3 mg/kg/day | | |
| Formula IA | 4 weeks | −17.2 ± 8.7° | −27.4 ± 27.2° |
| | 2 × 1.5 mg/kg/day | | |

Data expressed as mean ± SEM except for °(two-sided confidence interval)
**p < 0.01
*p < 0.05

1. A method of lowering the levels of Aβ₄₀ and Aβ₄₂ peptides in a mammal comprising administering to said mammal in need of treatment an effective amount of a monoamine neurotransmitter re-uptake inhibitor comprising a 2,3-disubstituted tropane moiety or a physiologically acceptable acid addition salt thereof.

2. A method according to claim 1, wherein said monoamine neurotransmitter re-uptake inhibitor is a compound of formula (I1) 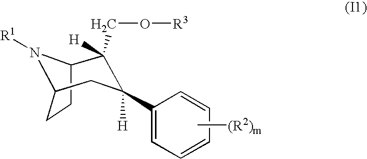
wherein
R¹ represents a hydrogen atom or a C₁₋₆ alkyl group;
R² represents a halogen atom or a CF₃ or cyano group;
R³ represents a hydrogen atom or a C₁₋₆ alkyl or C₃₋₆-cycloalkyl-C₁₋₃-alkyl group; and
m is 0 or an integer from 1 to 3.

3. A method according to claim 1, wherein said monoamine neurotransmitter re-uptake inhibitor is a compound of formula (IA) or (IB)

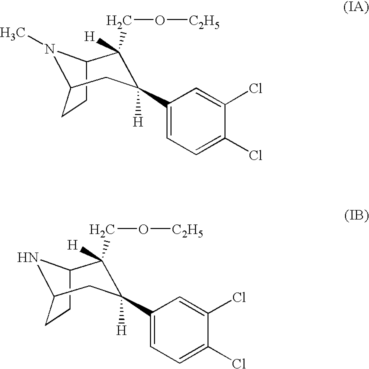

4. A method according to claim 1 for the treatment or prophylaxis of diseases associated with the formation of diffuse and senile plaques.

5. A method according to claim 1 for the treatment or prophylaxis of diseases associated with the formation of Aβ₄₀- or Aβ₄₂-containing plaques.

6. A method according to claim 1 for the treatment or prophylaxis of amyloidosis associated with the formation of Aβ₄₀ or Aβ₄₂.

7. A method according to claim 1 for the treatment or prophylaxis of brain amyloidosis.

8. A method according to claim 1 for the treatment or prophylaxis of vascular amyloidosis or age related amyloidosis.

9. A method according to claim 1 for the prevention of the progression of Alzheimer disease in a patient suffering from said disease.
